(19) United States
(12) Patent Application Publication
(10) Pub. No.: US 2017/0135417 A1
(43) Pub. Date: May 18, 2017

(54) AIR POCKET SENSOR

(71) Applicant: Flex Ltd., Singapore (SG)

(72) Inventor: Armando J. Lucrecio, Fremont, CA (US)

(21) Appl. No.: 15/350,407

(22) Filed: Nov. 14, 2016

Related U.S. Application Data
(60) Provisional application No. 62/254,515, filed on Nov. 12, 2015.

(51) Int. Cl. A41D 1/00; G01L 1/00; A61B 5/00; G01K 13/00; A43B 3/00; A61B 5/024

(52) U.S. Cl. CPC A41D 1/005; A43B 3/0005; A61B 5/024; A61B 5/4266; G01K 13/002; G01L 1/00; A63B 2220/803; A63B 2220/40

(57) ABSTRACT

Embodiments of the disclosure are directed to air pocket sensors and wearable articles including air pocket sensors. According to one embodiment, an air pocket sensor can comprise a sensor enclosure comprising a substantially disc-shaped enclosure base and a substantially disc-shaped enclosure cap of a substantially same size as and joined with the enclosure base. The enclosure cap can comprise a domed portion forming an air pocket within the sensor enclosure. An electronic sensor can be disposed within the air pocket of the sensor enclosure. An electrical conductor can be connected with the electronic sensor and can extend through the sensor enclosure. The electronic sensor can comprise, for example, a pressure sensor, a temperature sensor, a medical sensor, etc. In some cases, the enclosure can be substantially air tight.

### CROSS REFERENCE TO RELATED APPLICATION

This application claims the benefit of and priority under 35 U.S.C. §119(e) to U.S. Provisional Patent Application Ser. No. 62/254,515 filed on Nov. 12, 2015, by Lucrecio and entitled “Air Pocket Pressure Sensor” of which the entire disclosure is incorporated herein by reference in its entirety.

### FIELD OF THE DISCLOSURE

Embodiments of the present disclosure relate generally to electronic sensors and more particularly to air pocket sensors and wearable articles including air pocket sensors.

### BACKGROUND

The use of wearable devices of different types has grown and continues to grow. For example, various fitness trackers, smart watches, and similar devices are increasingly common and becoming more popular. Additionally, wearable technologies are increasingly being integrated into various other articles such as articles of clothing. These wearable technologies often rely on sensors such as force sensitive resistor and/or force sensitive capacitive sensors embedded in the article. However, these technologies require a multiplexer to read the sensors and afterwards a post-processing is needed to transform the signal to meaningful data. This is high cost and very expensive power wise and requires a high data rate system. Furthermore, these technologies are not suitable for random or irregular sensor disposition on the target surface.

### BRIEF SUMMARY

Embodiments of the disclosure are directed to air pocket sensors and wearable articles including air pocket sensors. Embodiments of the disclosure provide sensors, such as pressure sensors, temperature sensors, etc., that can be discretely positioned individually or as an array of sensors spread within a wearable article such as a shoe or other article of clothing. For example, air pocket sensors as described herein can be located at one or more points of interest within the article. According to one embodiment, each air pocket sensor can include a single MicroElectricalMechanical Systems (MEMS) component device positioned within an air pocket formed by an enclosure of the sensor. These devices can provide a clean output with no need of post processing, and can be disposed randomly or irregularly around any surface. Accordingly, the sensors described herein can allow inter-integrated circuit (I2C) communication that eliminates the typical analog/digital conversion for conventional sensors. Additionally, the precision and sensitivity of this measurement can be higher and easier to process.

According to one embodiment, an air pocket sensor can comprise a sensor enclosure comprising a substantially disc-shaped enclosure base and a substantially disc-shaped enclosure cap of a substantially same size as and joined with the enclosure base. The enclosure cap can comprise a domed portion forming an air pocket within the sensor enclosure. An electronic sensor can be disposed within the air pocket of the sensor enclosure. An electrical conductor can be connected with the electronic sensor and can extend through the sensor enclosure. The electronic sensor can comprise, for example, a pressure sensor, a temperature sensor, a medical sensor, etc. In some cases, the enclosure can be substantially air tight.

According to another embodiment, a sensor array can comprise a plurality of air pocket sensors. Each air pocket sensor of the sensor array can comprise a sensor enclosure comprising a substantially disc-shaped enclosure base and a substantially disc-shaped enclosure cap of a substantially same size as and joined with the enclosure base. The enclosure cap can comprise a domed portion forming an air pocket within the sensor enclosure. An electronic sensor can be disposed within the air pocket of the sensor enclosure. An electrical conductor can be connected with the electronic sensor and can extend through the sensor enclosure. The electronic sensor can comprise, for example, a pressure sensor, a temperature sensor, a medical sensor, etc. In some cases, the enclosure can be substantially air tight. In other cases, the air pocket of at least one air pocket sensor of the sensor array can be at least partially open to the air pocket of at least one other air pocket sensor of the sensor array.

According to yet another embodiment, a sensor embedded fabric can comprise a fabric base layer, a sensor array adjacent to the fabric base layer, and a mesh screen layer substantially adjacent to the sensor array. The sensor array can comprise a plurality of air pocket sensors. Each air pocket sensor of the sensor array can comprise a sensor enclosure comprising a substantially disc-shaped enclosure base and a substantially disc-shaped enclosure cap of a substantially same size as and joined with the enclosure base. The enclosure cap can comprise a domed portion forming an air pocket within the sensor enclosure. An electronic sensor can be disposed within the air pocket of the sensor enclosure. An electrical conductor can be connected with the electronic sensor and can extend through the sensor enclosure. The electronic sensor can comprise, for example, a pressure sensor, a temperature sensor, a medical sensor, etc. The sensor embedded fabric can further comprise a flexible and breathable punched layer between the fabric base layer and the mesh screen layer. The punched layer can comprise a plurality of holes into which the domed portion of the sensor enclosure of each air pocket sensor of the sensor array is inserted. In some cases, the enclosure of each air pocket sensor of the sensor array is substantially air tight. In other cases, the air pocket of at least one air pocket sensor of the sensor array can be at least partially open to the air pocket of at least one other air pocket sensor of the sensor array. The sensor embedded fabric can comprise at least a portion of an article of clothing.

### BRIEF DESCRIPTION OF THE DRAWINGS

FIG. 1 is a diagram illustrating an isometric view of an assembled air pocket sensor according to one embodiment.

FIG. 2 is a diagram illustrating an exploded isometric view of an air pocket sensor according to one embodiment.

FIG. 3 is a diagram illustrating an exploded isometric view of a wearable product in which air pocket sensors are embedded according to one embodiment.

FIG. 4 is a diagram illustrating an isometric view of an array of air pocket sensors according to one embodiment.

FIG. 5 is a diagram illustrating an exploded isometric view of a sensor embedded fabric according to one embodiment.

FIG. 6 is a diagram illustrating an isometric view of a sensor embedded fabric according to one embodiment.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

In the following description, for the purposes of explanation, numerous specific details are set forth in order to provide a thorough understanding of various embodiments disclosed herein. It will be apparent, however, to one skilled in the art that various embodiments of the present disclosure may be practiced without some of these specific details. The ensuing description provides exemplary embodiments only, and is not intended to limit the scope or applicability of the disclosure. Furthermore, to avoid unnecessarily obscuring the present disclosure, the preceding description omits a number of known structures and devices. This omission is not to be construed as a limitation of the scopes of the claims. Rather, the ensuing description of the exemplary embodiments will provide those skilled in the art with an enabling description for implementing an exemplary embodiment. It should however be appreciated that the present disclosure may be practiced in a variety of ways beyond the specific detail set forth herein.

While the exemplary aspects, embodiments, and/or configurations illustrated herein show the various components of the system collocated, certain components of the system can be located remotely, at distant portions of a distributed network, such as a LAN and/or the Internet, or within a dedicated system. Thus, it should be appreciated, that the components of the system can be combined in to one or more devices or collocated on a particular node of a distributed network, such as an analog and/or digital telecommunications network, a packet-switch network, or a circuit-switched network. It will be appreciated from the following description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

A “computer readable signal” medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable medium may be transmitted using any appropriate medium, including but not limited to wireless, wireline, optical fiber cable, RF, etc., or any suitable combination of the foregoing.

The terms “determine,” “calculate,” and “compute,” and variations thereof, as used herein, are used interchangeably and include any type of methodology, process, mathematical operation or technique.

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the disclosure, brief description of the drawings, detailed description, abstract, and claims themselves.

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Any combination of one or more computer readable medium(s) may be utilized. The computer readable medium may be a computer readable signal medium or a computer readable storage medium.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the disclosed embodiments, configurations, and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

Examples of the processors as described herein may include, but are not limited to, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22nm Haswell, Intel® Core® i5-3570K 22nm Ivy Bridge, the AMD® FX™ family of processors, AMD® FX-4300, FX-6300, and FX-8350 32nm Vishera, AMD® Kaveri processors, Texas Instruments® Jacinto C6000TM automotive infotainment processors, Texas Instruments® OMAP™ automotive-grade mobile processors, ARM® ^{Cortex™-M} processors, ARM® Cortex-A and ARM926EJ-S™ processors, other industry-equivalent processors, and may perform computational functions using any known or future-developed standard, instruction set, libraries, and/or architecture.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

Embodiments of the disclosure are directed to air pocket sensors and wearable articles including air pocket sensors. Embodiments of the disclosure provide sensors, such as pressure sensors, temperature sensors, etc., that can be discretely positioned individually or as an array of sensors spread within a wearable article such as a shoe or other article of clothing. For example, air pocket sensors as described herein can be located at one or more points of interest within the article. According to one embodiment, each air pocket sensor can include a single MicroElectricalMechanical Systems (MEMS) component device positioned within an air pocket formed by an enclosure of the sensor. These devices can provide a clean output with no need of post processing, and can be disposed randomly or irregularly around any surface. Accordingly, the sensors described herein can allow inter-integrated circuit (I2C) communication that eliminates the typical analog/digital conversion for conventional sensors. Additionally, the precision and sensitivity of this measurement can be higher and easier to process. Various additional details of embodiments of the present disclosure will be described below with reference to the figures. While the flowcharts will be discussed and illustrated in relation to a particular sequence of events, it should be appreciated that changes, additions, and omissions to this sequence can occur without materially affecting the operation of the disclosed embodiments, configuration, and aspects.

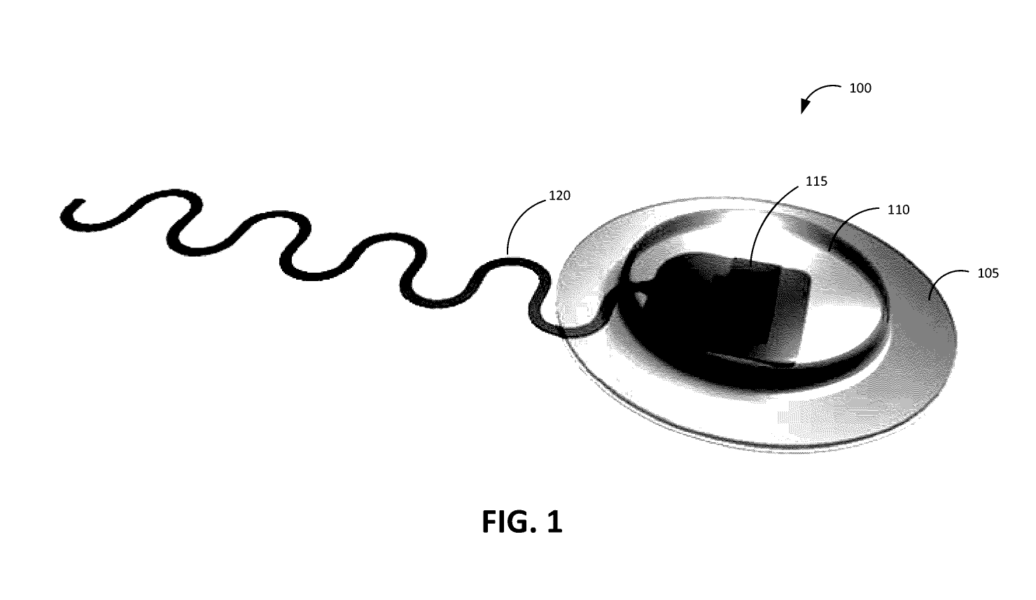

FIG. 1 is a diagram illustrating an isometric view of an assembled air pocket sensor according to one embodiment. As illustrated in this example, the air pocket sensor 100, according to one embodiment, can comprise a sensor enclosure 105 or covering having a disc shape with a domed portion forming an air pocket 110. An electronic sensor 115 can be positioned and installed within the air pocket 110. An electrical conductor 120 can extend from the electronic sensor 115 through the sensor enclosure 105 of the air pocket sensor 100. The conductor 120 can allow the air pocket sensor 100 to be connected with a microprocessor or other controller to which the electronic sensor 115 can provide a signal indicating a measurement of a physical condition detected by the electronic sensor 115. In some cases and as will be described in greater detail below, one or more air pocket sensors 100 can be connected together to form an array. In such cases, the conductor 120 can allow a plurality of air pocket sensors to be electrically connected with the microprocessor or controller.

According to one embodiment, the electronic sensor 115 can comprise a pressure sensor such as a piezoelectric, piezo-resistive, capacitive, electromagnetic, or other type of pressure sensor. In other cases, the electronic sensor 115 can comprise another type of sensor including but not limited to a temperature sensor, such as a thermistor, resistance thermometer, silicone bandgap temperature sensor, etc., a motion sensor, an accelerometer, or a medical sensor such as perspiration sensor or heart rate sensor. Various other types of sensors may be implemented as the electronic sensor 115 within the air pocket sensor 100 in various embodiments. According to one embodiment, each air pocket sensor 100 can include a single MicroElectricalMechanical Systems (MEMS) component device sensor 115 positioned within the air pocket 110 formed by the enclosure 105 of the sensor 100. These devices can provide a clean output with no need of post processing, and can be disposed randomly or irregularly around any surface. Accordingly, the sensors 100 described herein can allow inter-integrated circuit (I2C) communication that eliminates the typical analog/digital conversion for conventional sensors. Additionally, the precision and sensitivity of this measurement can be higher and easier to process.

By using an enclosure 105 and air pocket 110 around a MEMS sensor 115, a more precise measurement, e.g., a pressure measurement, is achievable over other sensors. This is due in part to MEMS devices being very precise devices while other implementations, e.g., resistor and capacitor based, need constant calibration to keep minimal precision, and they are very sensitive to temperature and humidity changes. MEMS sensors can also provide temperature and humidity information simultaneously. These sensors are also low power, low cost and low data rate compared with other solutions in the market.

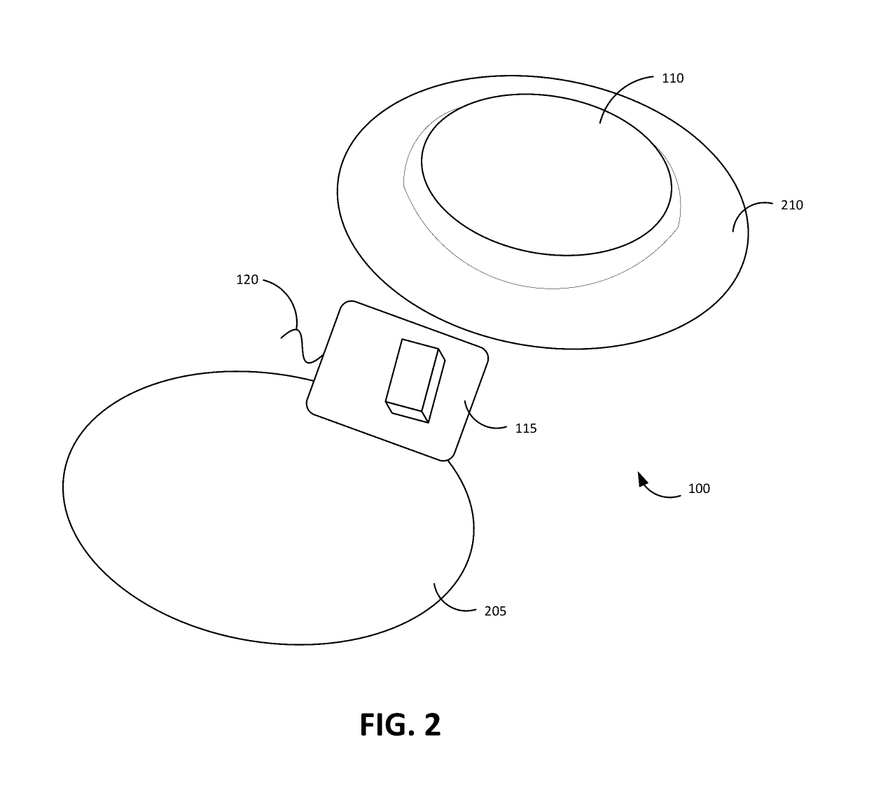

FIG. 2 is a diagram illustrating an exploded isometric view of an air pocket sensor according to one embodiment. This example illustrates the air pocket sensor 100 as described above with reference to FIG. 1. As can be seen in this view, the air pocket sensor 100 can comprise two portions forming the sensor enclosure 105 described above. More specifically, the air pocket sensor 100 can comprise an enclosure base 205 and an enclosure cap 210. According to one embodiment, both the enclosure base 205 and enclosure cap 210 can be substantially disc-shaped as illustrated here. However, in other embodiments and in different implementations, the enclosure base 205 and/or enclosure cap 210 can be of different shapes thereby allowing the assembled air pocket sensor 100 to be, for example, substantially square, rectangular, polygonal, hexagonal, etc. Regardless of the exact overall shape of the air pocket sensor 100, the enclosure cap 210 can include a domed or raised portion forming the air pocket 110 into which the electronic sensor will be positioned and mounted once the air pocket sensor 100 is assembled. Materials suitable for the enclosure include, but are not limited to, silicone, plastic, polymers, water absorbent polymers material or any other flexible material and stretchable material.

To assemble the air pocket sensor 100, the electronic sensor 115 can be positioned between the enclosure base 205 and enclosure cap 210 so that the enclosure base aligns 205 with the enclosure cap 210 and the electronic sensor 115 aligns with the air pocket 110 formed by the domed or raised portion of the enclosure cap 210. The enclosure base 205 and enclosure cap 210 can then be physically joined by an adhesive layer (not shown here) between the two, by being fused together by heat or pressure, or by other means. According to one embodiment, the joining or fusing of the enclosure base 205 and enclosure cap 210 can be substantially air tight such that the resulting sensor enclosure 105 creates a sealed environment within the air pocket 110. In other cases, the joining or fusing of the enclosure base 205 and enclosure cap 210 may not be completely or substantially air tight thereby allowing some air flow in and out of the air pocket 110. In such cases, the air flow into and out of the air pocket may be metered or valved, e.g., based on a size of an orifice or opening between the enclosure base 205 and enclosure cap 210. In some cases, such as when a plurality of air pocket sensors 100 are assembled into an array as will be described in greater detail below, the air pocket 110 of one air pocket sensor 100 may be vented to or allow air flow to and from the air pocket 110 of one or more adjacent air pocket sensors 100.

Stated another way, an air pocket sensor 100 can comprise a sensor enclosure 105 comprising a substantially disc-shaped enclosure base 205 and a substantially disc-shaped enclosure cap 210 of a substantially same size as and joined with the enclosure base 205. The enclosure cap 210 can comprise a domed portion forming an air pocket 110 within the sensor enclosure. An electronic sensor 115 can be disposed within the air pocket 1150 of the sensor enclosure 105. An electrical conductor 120 can be connected with the electronic sensor 115 and can extend through the sensor enclosure 105. The electronic sensor 115 can comprise, for example, a pressure sensor, a temperature sensor, a medical sensor, etc. In some cases, the enclosure 105 can be substantially air tight.

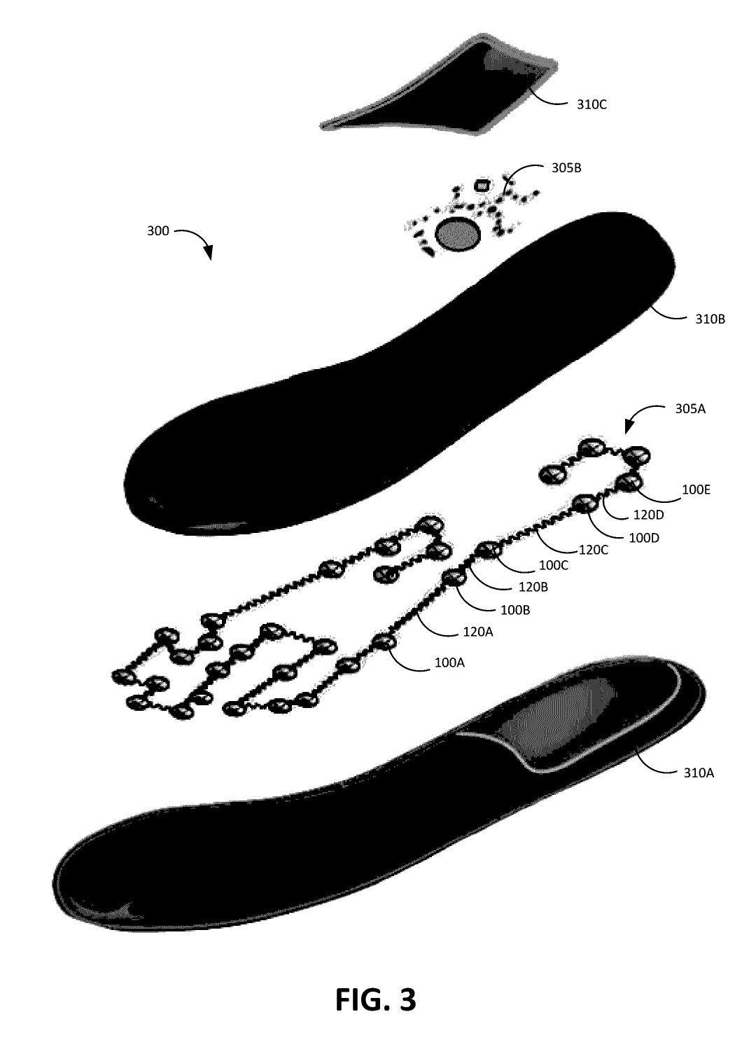

FIG. 3 is a diagram illustrating an exploded isometric view of a wearable product in which air pocket sensors are embedded according to one embodiment. More specifically, this example illustrates a shoe insert 300 or insole into which a plurality of air pocket sensors 100A-100E can be embedded. As illustrated here, the shoe insert 300 can comprise a shoe insert bottom portion 310A and a shoe insert top portion 310B. A sensor array 305A can be located between the shoe insert bottom 310A and shoe insert top 310B. The sensor array 305A can comprise a plurality of air pocket sensors 100A, 100B, 100C, 100D, and 100E as described above. Also as shown here, the air pocket sensors 100A-100E of the sensor array 305A can be connected via the conductors 120A, 120B, 120C, and 120D of the individual air pocket sensors 100A-100E. Furthermore, the sensor array 305A can be connected with a microprocessor, controller or other integrated circuit (not shown here) located in the shoe insert 300 or elsewhere. It should be understood that any number of air pocket sensors may be utilized in various embodiments depending upon the exact implementation.

To assemble the shoe insert 300, the sensor array 305A can be positioned between the shoe insert bottom 310A and shoe insert top 310B so that the shoe insert bottom 310A aligns with the shoe insert top 310B and the sensor array 305A and individual air pocket sensors 100A-100E thereof align with the positions within the shoe insert 300 that the individual air pocket sensors 100A-100E are intended to measure or monitor. The shoe insert bottom 310A and shoe insert top 310B can then be physically joined by an adhesive layer (not shown here) between the two, by being fused together by heat or pressure, or by other means.

Additionally or alternatively, the shoe insert 300 can comprise a sensor array 305B positioned on top of the shoe insert 300 and enclosed by a cover 310C which can be affixed to the shoe insert top 310B by an adhesive or by other means. According to one embodiment, sensor array 305B may be implemented in addition to sensor array 305A. For example, one sensor array 305A may be positioned to measure physical characteristics, e.g., pressure, temperature, etc., at certain locations of the shoe insert 300 such as the toes and/or arches while sensor array 305B may be positioned to measure physical characteristics, e.g., pressure, temperature, etc., at certain other locations of the shoe insert 300 such as the heal. In other cases, one sensor array 305A can comprise one type of air pocket sensor, e.g., pressure sensors, while another sensor array 305B can comprise a different type of air pocket sensor, e.g., temperature sensors. Alternatively, sensor array 305B may be implemented instead of or as an alternative to sensor array 305A. In such cases, the shoe insert 300 may comprise only one portion, e.g., shoe insert top 310B, with the sensor array 305B and cover 310C. Other alternative configurations are contemplated and considered to be within the scope of the present disclosure.

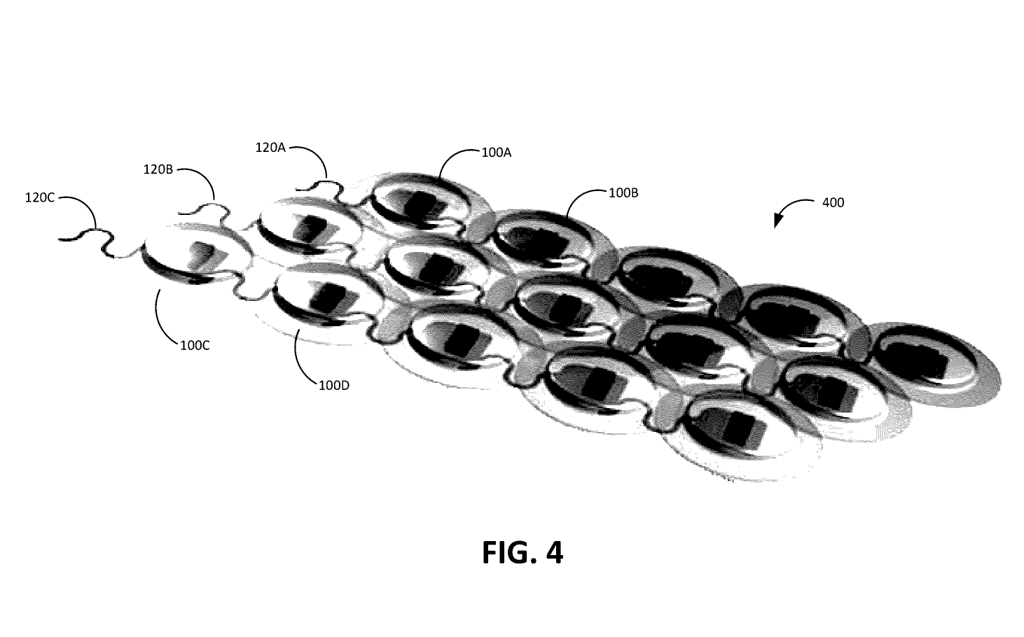

FIG. 4 is a diagram illustrating an isometric view of an array of air pocket sensors according to one embodiment. As noted above and illustrated here, a plurality of air pocket sensors 100A, 100B, 100C, and 100D can be arranged into a sensor array 400. In this example, the air pocket sensors 100A-100D are arranged in a grid pattern. Such a grid can have any number of different dimensions depending upon the exact implementation. In other cases, the air pocket sensors 100A-100D can be arranged in a different configuration, i.e., other than a grid, and in different patterns. Regardless of the exact configuration, the air pocket sensors 100A-100D can be physically joined together, e.g., by adhesive, fused together by het or pressure, or by other means. The sensors can also electrically connected with one or more other sensors and one or more conductors 120A, 120B, and 120C can extend from the sensor array 400 for connection to a microprocessor or other controller. As noted above, each air pocket sensor 100A-100D of the sensor array 400 can be sealed and substantially air tight. In other cases, each air pocket sensor 100A-100D may be vented to one or more neighboring air pocket sensors, e.g., through a valve, vent, or air passage between air pockets of the individual air pocket sensors. In this way, pressure can be equalized across the sensor array 400 or portions of the sensor array 400. Such a sensor array 400 can be embedded into any number of wearable articles, e.g., shoes, pants, shirts, jackets, hats, gloves, helmets, other protective sports equipment, etc. In some cases, such a sensor array 400 may be embedded within a fabric that can be used to produce such articles.

Stated another way, a sensor array 400 can comprise a plurality of air pocket sensors 100A-100D. Each air pocket sensor 100 of the sensor array 400 can comprise a sensor enclosure 105 comprising a substantially disc-shaped enclosure base 205 and a substantially disc-shaped enclosure cap 210 of a substantially same size as and joined with the enclosure base 205. The enclosure cap 210 can comprise a domed portion forming an air pocket 110 within the sensor enclosure 105. An electronic sensor 115 can be disposed within the air pocket 110 of the sensor enclosure 105. An electrical conductor 120 can be connected with the electronic sensor 115 and can extend through the sensor enclosure 105. The electronic sensor 115 can comprise, for example, a pressure sensor, a temperature sensor, a medical sensor, etc. In some cases, the enclosure 105 can be substantially air tight. In other cases, the air pocket 110 of at least one air pocket sensor 100A of the sensor array 400 can be at least partially open to the air pocket 110 of at least one other air pocket sensor 100B of the sensor array 400.

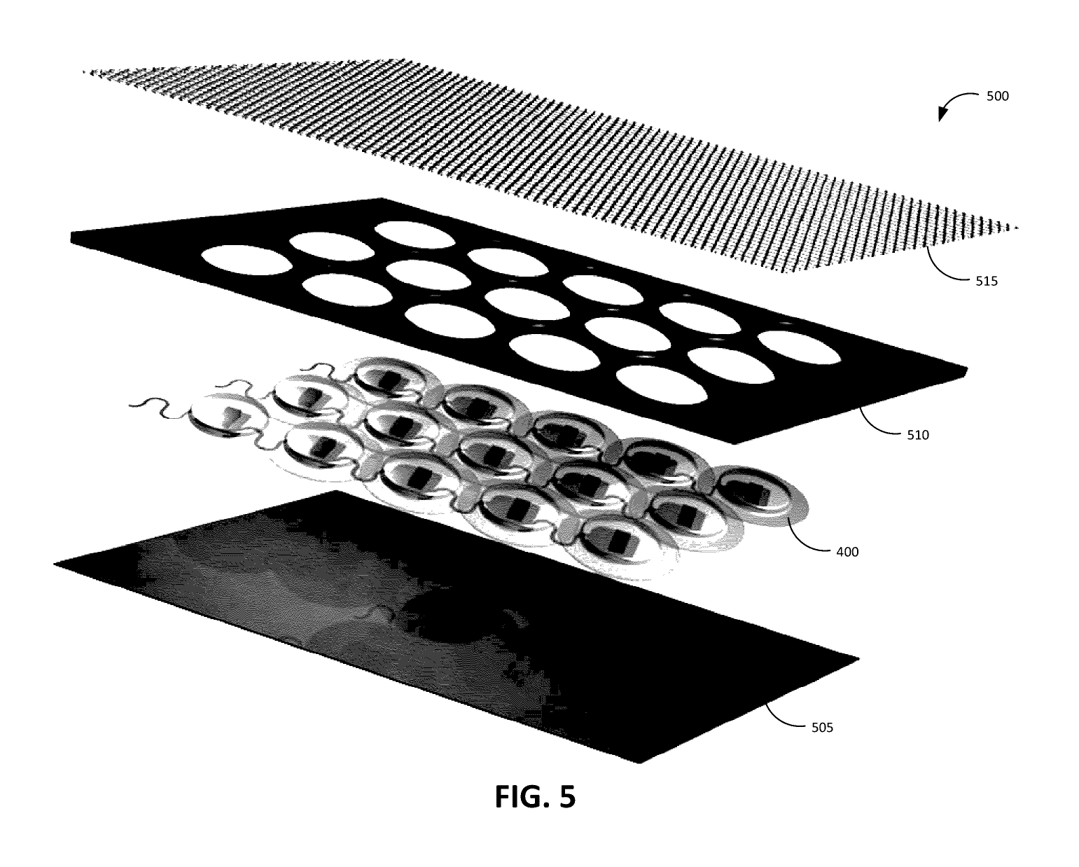

FIG. 5 is a diagram illustrating an exploded isometric view of a sensor embedded fabric according to one embodiment. As illustrated here, a sensor embedded fabric 500 can comprise a sensor array 400 such as described above. The sensor embedded fabric 500 can also comprise a base layer 505, a punched layer 510 and a screen layer 515 or top layer. Generally speaking, the base layer 505 can comprise a variety of different flexible and breathable fabrics or materials and can, in some cases, comprise the outside of a garment, i.e., away from the wearers body, when the fabric is fashioned into the garment. The punched layer 510 may, in some cases, be an optional layer. If used, the punched layer 510 can comprise a variety of different flexible and breathable fabrics or materials with a number of alignment holes into which the individual air pocket sensors of the sensor array 400 will fit when the fabric is assembled. The screen layer 515 can comprise a flexible and breathable screen or mesh material and can, in some cases, comprise the inside of a garment, i.e., closest to the wearers body, when the fabric is fashioned into the garment.

To assemble the sensor embedded fabric 500, the sensor array 400 can be positioned between the base layer 505 and punched layer 510, if used, so that the individual air pocket sensors of the sensor array 400 align with the alignment holes in the punched layer. The screen layer 515 can then be laid onto the punched layer 510 or onto the sensor array 400 if the punched layer 510 is omitted. The base layer 505, sensor array 400, punched layer 510, if any, and screen layer 515 can then be physically joined by an adhesive layer (not shown here) between each layer, by stitching, sewing, or weaving the layers 505, 510, and 515 together, or by other means.

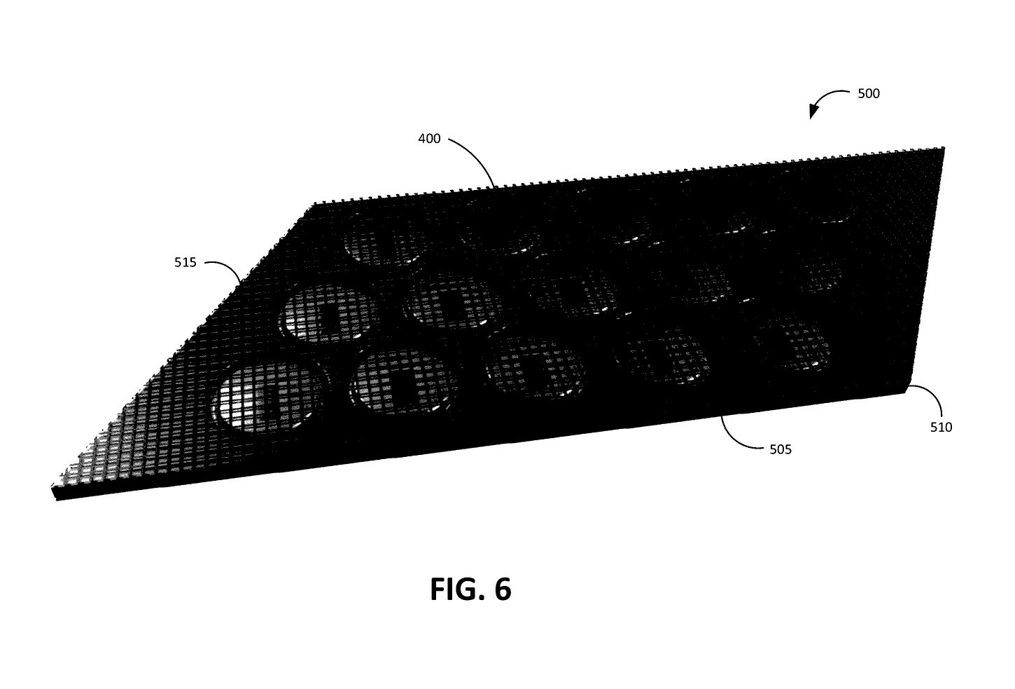

FIG. 6 is a diagram illustrating an isometric view of a sensor embedded fabric according to one embodiment. More specifically, this example illustrates the sensor embedded fabric 500 described above after the base layer 505, sensor array 400, punched layer 510, and screen layer 515 have been assembled and joined together. It should be noted that the size and dimensions of the fabric 500 and the size or dimensions of the sensor array 400 within the fabric 500 can vary significantly, both in total and relative to each other. That is, while this example illustrates the sensor array 400 substantially covering the dimensions of the fabric 500, the dimensions of the sensor array 400 may, in some cases, be substantially smaller than the fabric 500. In some cases, the fabric 500 may be rather large compared to the size of the sensor array 400. In such cases, the fabric 500 may include more than one sensor array 400 embedded therein. For example, a plurality of sensor arrays 400 may be embedded in the fabric 500 at various different intervals or positions. In this way, the sensor arrays 400 can provide coverage across the fabric 500 but covering the entire fabric 500. In other cases, the sensor arrays 400 may be placed at certain locations which will be used at certain positions on the wearer's body when the fabric is assembled into an article of clothing or other wearable product. Numerous alternative arrangements are contemplated an considered to be within the scope of the present disclosure.

Stated another way, a sensor embedded fabric 500 can comprise a fabric base layer 505, a sensor array 400 adjacent to the fabric base layer 505, and a mesh screen layer 515 substantially adjacent to the sensor array 400. The sensor array 400 can comprise a plurality of air pocket sensors 100. Each air pocket sensor 100 of the sensor array 400 can comprise a sensor enclosure 105 comprising a substantially disc-shaped enclosure base 205 and a substantially disc-shaped enclosure cap 210 of a substantially same size as and joined with the enclosure base 205. The enclosure cap 205 can comprise a domed portion forming an air pocket 110 within the sensor enclosure 105. An electronic sensor 115 can be disposed within the air pocket 110 of the sensor enclosure 105. An electrical conductor 120 can be connected with the electronic sensor 115 and can extend through the sensor enclosure 10. The electronic sensor 115 can comprise, for example, a pressure sensor, a temperature sensor, a medical sensor, etc. The sensor embedded fabric 500 can further comprise a flexible and breathable punched layer 510 between the fabric base layer 505 and the mesh screen layer 515. The punched layer 510 can comprise a plurality of holes into which the domed portion of the sensor enclosure 105 of each air pocket sensor 100 of the sensor array 400 is inserted. In some cases, the enclosure 105 of each air pocket sensor 100 of the sensor array 400 is substantially air tight. In other cases, the air pocket 110 of at least one air pocket sensor 100 of the sensor array 400 can be at least partially open to the air pocket 110 of at least one other air pocket sensor 100 of the sensor array 400. The sensor embedded fabric 500 can comprise at least a portion of an article of clothing.

The present disclosure, in various aspects, embodiments, and/or configurations, includes components, methods, processes, systems, and/or apparatus substantially as depicted and described herein, including various aspects, embodiments, configurations embodiments, sub combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An air pocket sensor comprising:
a sensor enclosure comprising:
a substantially disc-shaped enclosure base, and
a substantially disc-shaped enclosure cap of a substantially same size as and joined with the enclosure base, the enclosure cap comprising a domed portion forming an air pocket within the sensor enclosure; and
an electronic sensor disposed within the air pocket of the sensor enclosure.

2. The air pocket sensor of claim 1, further comprising an electrical conductor connected with the electronic sensor and extending through the sensor enclosure.

3. The air pocket sensor of claim 1, wherein the electronic sensor comprises a pressure sensor.

4. The air pocket sensor of claim 1, wherein the electronic sensor comprises a temperature sensor.

5. The air pocket sensor of claim 1, wherein the electronic sensor comprises a medical sensor.

6. The air pocket sensor of claim 1, wherein the enclosure is substantially air tight.

7. A sensor array comprising a plurality of air pocket sensors, each air pocket sensor of the sensor array comprising:
a sensor enclosure comprising:
a substantially disc-shaped enclosure base, and
a substantially disc-shaped enclosure cap of a substantially same size as and joined with the enclosure base, the enclosure cap comprising a domed portion forming an air pocket within the sensor enclosure;
an electronic sensor disposed within the air pocket of the sensor enclosure; and
an electrical conductor connected with the electronic sensor and extending through the sensor enclosure.

8. The sensor array of claim 7, wherein the electronic sensor of at least one air pocket sensor of the sensor array comprises a pressure sensor.

9. The sensor array of claim 7, wherein the electronic sensor of at least one air pocket sensor of the sensor array comprises a temperature sensor.

10. The sensor array of claim 7, wherein the electronic sensor of at least one air pocket sensor of the sensor array comprises a medical sensor.

11. The sensor array of claim 7, wherein the enclosure of each air pocket sensor of the sensor array is substantially air tight.

12. The sensor array of claim 7, wherein the air pocket of at least one air pocket sensor of the sensor array is at least partially open to the air pocket of at least one other air pocket sensor of the sensor array.

13. A sensor embedded fabric comprising:
a fabric base layer;
a sensor array adjacent to the fabric base layer, the sensor array comprising a plurality of air pocket sensors, each air pocket sensor of the sensor array comprising:
a sensor enclosure comprising:
a substantially disc-shaped enclosure base, and
a substantially disc-shaped enclosure cap of a substantially same size as and joined with the enclosure base, the enclosure cap comprising a domed portion forming an air pocket within the sensor enclosure;
an electronic sensor disposed within the air pocket of the sensor enclosure; and
an electrical conductor connected with the electronic sensor and extending through the sensor enclosure; and
a mesh screen layer substantially adjacent to the sensor array.

14. The sensor embedded fabric of claim 13, further comprising a flexible and breathable punched layer between the fabric base layer and the mesh screen layer, the punched layer comprising a plurality of holes into which the domed portion of the sensor enclosure of each air pocket sensor of the sensor array is inserted.

15. The sensor embedded fabric of claim 13, wherein the electronic sensor of at least one air pocket sensor of the sensor array comprises a pressure sensor.

16. The sensor embedded fabric of claim 13, wherein the electronic sensor of at least one air pocket sensor of the sensor array comprises a temperature sensor.

17. The sensor embedded fabric of claim 13, wherein the electronic sensor of at least one air pocket sensor of the sensor array comprises a medical sensor.

18. The sensor embedded fabric of claim 13, wherein the enclosure of each air pocket sensor of the sensor array is substantially air tight.

19. The sensor embedded fabric of claim 13, wherein the air pocket of at least one air pocket sensor of the sensor array is at least partially open to the air pocket of at least one other air pocket sensor of the sensor array.

20. The sensor embedded fabric of claim 13, wherein the sensor embedded fabric comprises at least a portion of an article of clothing.
